(19) United States
(12) Patent Application Publication
(10) Pub. No.: US 2006/0173527 A1
(43) Pub. Date: Aug. 3, 2006

(54) Stent for insertion and expansion in a lumen

(76) Inventor: Frank Scherrible, Knichissle (DE)

Correspondence Address: MCDONALD HOPKINS CO., LPA, 2100 BANK ONE CENTER, 600 SUPERIOR AVENUE, E., CLEVELAND, OH, 44114-2653, US

(21) Appl. No.: 11/321,904

(22) Filed: Dec. 29, 2005

Related U.S. Application Data
(63) Continuation-in-part of application No. 11/039,190, filed on Jan. 20, 2005.

(30) Foreign Application Priority Data
Jan. 21, 2004 (DE) 102004003093.6-43

(51) Int. Cl. A61F 2/06

(52) U.S. Cl. 623/1.15

(57) ABSTRACT

A stent with at least two portions of a stent structure that are moved toward one another or away from each other upon expansion of the stent is characterized in that between the two portions there is at least one coupling arrangement by means of which, after a predefined movement of the two portions, these portions are coupled to one another in such a way that movement of the two portions away from one another and/or further movement of the two portions toward one another is substantially avoided.

### CROSS-REFERENCE TO RELATED APPLICATIONS

This application is a continuation-in-part of prior pending U.S. patent application Ser. No. 11/039,190 filed on Jan. 20, 2005, which claims priority from German Patent Application No. 10 2004 003093.6-43 filed on Jan. 21, 2004.

### FIELD OF INVENTION

The present invention relates generally to stents, and in particular, to a use of a stent for insertion and/or expansion in a lumen or for implantation in a living body, with at least two portions of a stent structure which are moved toward one another upon expansion of the stent.

### BACKGROUND

Stents of the abovementioned type are used to avoid collapse or occlusion of a lumen or channel of a living body, for example blood vessels, esophagus, urethra or renal ducts, by expansion of a substantially tubular wall structure of the stent inside the channel. The inserted stent can increase the cross section of flow of the lumen for a medium flowing therein or can keep this cross section of flow permanently large enough. Stents are also used as carriers for medicaments that permit at least local therapy in a channel of the body. Stents can additionally be used as aneurysm stents or endoprostheses for intracerebral vascular pouches or as intraluminal stents. Stents or spreading structures are also used as supports for implants.

The wall or stent structure of such stents have a large number of bridges that are connected to one another at bridge connectors or nodes. The bridges are made of a flexible material, for example nitinol or stainless steel, so that the stent as a whole has a slightly flexible wall structure. Stents that can be expanded by remote control are also known, these being actuated for example by means of a kind of Bowden cable. There are also self-expanding stents which are prestressed and automatically increase their diameter when suitably released.

To guarantee the desired cross section of flow of the lumen, it is desirable that a stent, once inserted and expanded, maintains its shape as permanently as possible and does not further expand or compress at a later stage. It would also be desirable if the stent could assume a predefined shape and could then retain this shape.

### SUMMARY OF THE INVENTION

It is, therefore, an object of the invention to improve a stent of the type mentioned above such that it has the abovementioned properties and such that the stent in particular assumes a permanently defined state of expansion.

According to the invention, this object is achieved by a stent, spreading structure, or support structure for insertion and/or expansion or support in a lumen, with at least two portions of a stent structure that are moved substantially toward one another upon expansion of the stent. Further, between the two portions of such stents, spreading structures, or support structures, there is at least one coupling arrangement by means of which, after at least one predefined movement of the two portions, these portions are coupled to one another in such a way that movement of the two portions away from one another is substantially avoided. Alternatively or in addition, the object is achieved by a stent, spreading structure, or support structure for insertion and/or expansion or support in a lumen, with at least two portions of a stent structure that are moved substantially toward one another upon expansion of the stent. Further, between the two portions of such stents, spreading structures, or support structures, there is at least one coupling arrangement by means of which, after at least one predefined movement of the two portions, these portions are coupled to one another in such a way that further movement of the two portions toward one another is substantially avoided.

Further, in accordance with the present invention, a coupling arrangement is provided between two portions of the stent, spreading structure, or support structure, these portions moving relative to one another upon expansion of the stent or of the spreading structure or support structure, which coupling arrangement fixes the portions in their offset position relative to one another and in this way no longer permits an undefined further expansion and/or compression of the stent. Moreover, because of the coupling arrangement according to the invention, the portions of the stent that are to be moved assume a predetermined position relative to one another so that the stent as a whole with its multiplicity of portions is likewise brought into a predefined form upon expansion and also remains in this form.

The coupling arrangement of the present invention is configured with a male plug element and female plug element, of which one in particular is arranged in a substantially fixed manner on a first of the two portions and the other is arranged in a substantially fixed manner on the second portion. The plug elements form a force-fit connection and/or form-fit connection by means of which the portions are fixed relative to one another in the compressed state of the stent. A further expansion or compression would deform the plug elements provided. The plug elements are therefore made rigid so that a certain residual flexibility of the stent is maintained in the expanded state, but such that the desired dimensional accuracy is achieved in this expanded state.

The coupling arrangement is formed with two bridge halves that are oriented substantially coaxially and have mutually facing end areas. The male plug element is formed on a first end area and the female plug element is formed on the second end area. The bridge halves can be cut out from a single bridge during the production of the stent, and the male plug element and the female plug element can be formed on them.

To ensure that the generally large number of individual coupling arrangements of a stent can all be closed without difficulty during expansion of the stent, the individual coupling arrangement can be configured with a guide element with which the male plug element is guided into the female plug element when the two portions are moved toward one another.

The coupling arrangement is also formed with two bridge halves that are oriented substantially coaxially and that have a guide rod, on the one hand, and a guide groove, on the other hand, arranged as guide element at mutually facing end areas. During production of the support bridges of the stent structure, the guide rod and the guide groove can be cut out particularly inexpensively from the material of the stent (e.g., a shape-memory material, in particular nitinol), for example by a laser welding process. The axis of the two bridge halves can advantageously extend substantially parallel to the longitudinal axis of the stent, since in this way it is possible to ensure that, during expansion of the stent, the guide rod remains virtually unbent, and instead is merely offset together with the guide groove. The guide rod therefore, does not emerge radially from the guide groove.

To safely avoid an undesired deformation of the stent, and in particular of its coupling arrangements, the two portions could already be guided relative to one another in the non-expanded state of the stent.

As a form-fit coupling of the bridge halves in a stent, the coupling arrangement can be configured with at least one hook element or groove/spring element that catches for example on an associated female plug element when the two portions are moved toward one another.

In the stent of the present invention, to ensure that the stent structure and the coupling arrangements provided therein can be cut out from the material of the stent in just one operation, the individual structural element of the stent structure is preferably in the form of at least four bridges that are connected pivotably or flexibly to one another at nodes. Additionally, the individual structural element, in the expanded state of the stent, form a closed (basically polygonal) ring shape, in particular a diamond shape, in the inside of which the coupling arrangement is disposed. Also, the coupling arrangement in this embodiment can be formed by means of two bridge halves that substantially form a diagonal inside the ring shape. Finally, the coupling arrangement of the stent can also be formed with two mutually facing bridge halves that are each individually articulated together with two adjacent support bridges at a node of the stent structure.

The stent of the present invention can be used as support for a heart valve. Additionally, the stent of the present invention can be used as support for a venous valve, a vena cava filter, a prostatic sphincter body and/or as an anti-reflux stent (stomach valve).

According to the present invention, a method for applying a stent for insertion and expansion in a lumen is disclosed. The method comprises the stent structure having at least two portions that are moved substantially toward one another upon expansion of the stent, and, upon expansion of the stent and after a predefined movement of the two portions. The two portions are coupled to one another by means of a coupling arrangement in such a way that movement of the two portions away from one another is substantially avoided and/or that further movement of the two portions toward one another is substantially avoided.

### DESCRIPTION OF THE DRAWINGS

Objects and advantages together with the operation of the invention may be better understood by reference to the following detailed description taken in connection with the following illustrations, wherein:

FIG. 1 shows a detail view of a structural element of a stent according to the present invention in the compressed state, where a coupling arrangement is disposed in the axial direction of the stent,

FIG. 2 shows the detail view from FIG. 1 in the expanded state,

FIG. 3 shows a developed view of a circumferential series of structural elements of a stent according to the present invention in the compressed state, and

FIG. 4 shows the developed view from FIG. 3 in the expanded state,

FIG. 5 shows a further stent in the compressed state, where the coupling arrangement is disposed in the circumferential direction of the stent,

FIG. 6 shows the stent from FIG. 5 in the expanded state,

FIG. 7 shows a further stent having a non-circular cross-section.

### DETAILED DESCRIPTION OF THE INVENTION

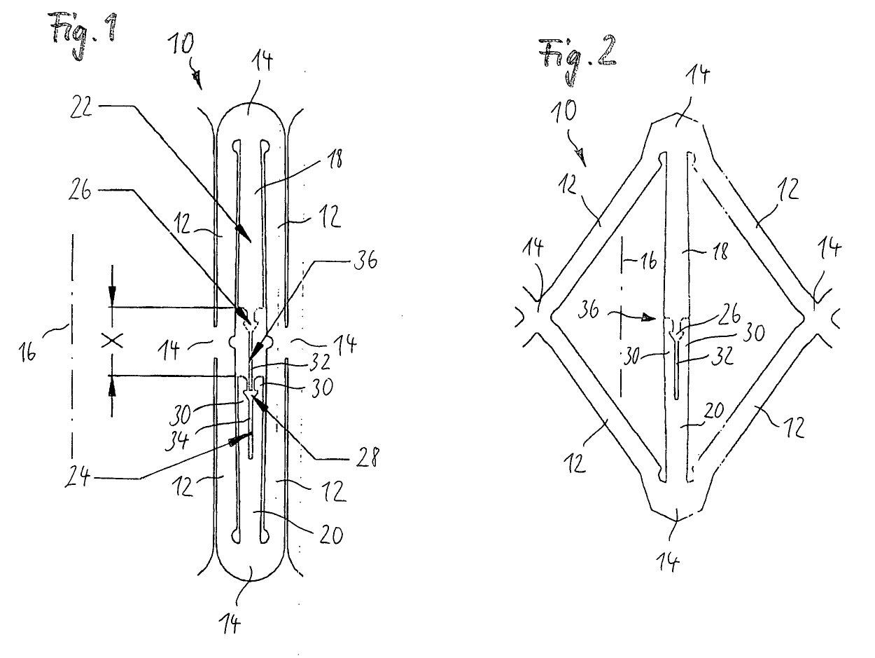

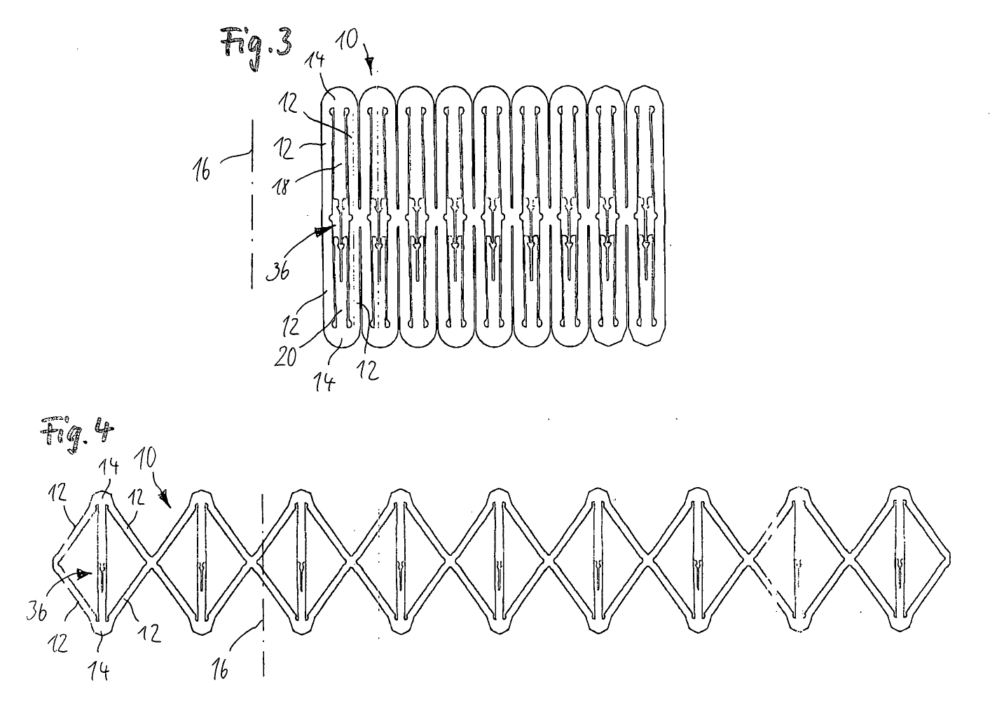

FIGS. 1 to 4 show portions of what is generally a substantially circular cylindrical stent, spreader, or support structure 10 for insertion and/or expansion or support in a lumen. The stent structure being composed of a multiplicity of support bridges 12. The support bridges 12 have nodes 14 at each end that serve as connecting portions for the support bridges 12.

In the compressed state of the stent 10, the support bridges 12 extend substantially in the direction of a longitudinal axis 16 of the stent 10 and in so doing form a kind of flattened diamond. In the inside of bridges 12 two coaxially disposed bridge halves 18 and 20 are arranged. At the ends remote from one another, the bridge halves 18 and 20 are each articulated on one of the nodes 14 and, at the end areas facing toward one another, they have a male plug element 22 or female plug element 24, respectively.

On the male plug element 22 and female plug element 24, respectively, mutually facing abutment surfaces are formed that, in the compressed state of the stent 10 as illustrated in FIGS. 1 and 3, are arranged at a defined distance or length of travel X from one another and are moved toward one another only upon expansion of the stent 10 through the movement of the associated nodes 14. During this movement, the plug connection or snap-fit connection formed by the male plug element 22 and female plug element 24 is brought together and locked in this position. This locking is achieved by means of one or more catch lugs 26 that are provided on the male plug element and that each lock into a corresponding recess 28 of the female plug element 24. To ensure that the catch lugs 26 can be guided into the female plug element 24, the latter is configured with two spring arms 30 in which the recesses 28 are formed. The catch lug or catch lugs 26 is/are preferably hook-shaped or double-hook-shaped or arrow-shaped, while the corresponding recess or recesses 28 has/have a substantially complementary shape.

In the case of a self-expanding stent 10, the expansion force should be greater than the counterforce of these spring arms 30 against the catch lugs 26. To achieve this, the last step of a heat treatment of such a stent 10 should bridge the difference from application of the stent to locking of its snap-fit connections. Moreover, the austenite finish temperature (Af temperature) of the stent material can be set so that handling of the stent 10 at normal ambient temperature, i.e., a temperature of ca. 20° C. to 25° C., is possible. Stents 10 expanding under external control are expanded manually or separately only upon application of the stent. In such stents 10, it must be ensured that the spring arms 30 on the snap-fit connections are not permanently or plastically deformed during expansion.

At the frontmost area of the male plug element 22, a guide rod 32 is also formed that, even in the compressed state of the stent 10, is guided in a guide groove 34 of the female plug element 24 and can be displaced further therein. In the locked state of the male plug element 22 and female plug element 24, the associated abovementioned abutment surfaces bear on one another, thus creating a locked coupling arrangement 36. With this coupling arrangement 36, the otherwise movable nodes 14 belonging to the bridge halves 18 and 20 are fixed relative to one another, and the support bridges 12 are locked in a substantially no longer deformable diamond shape.

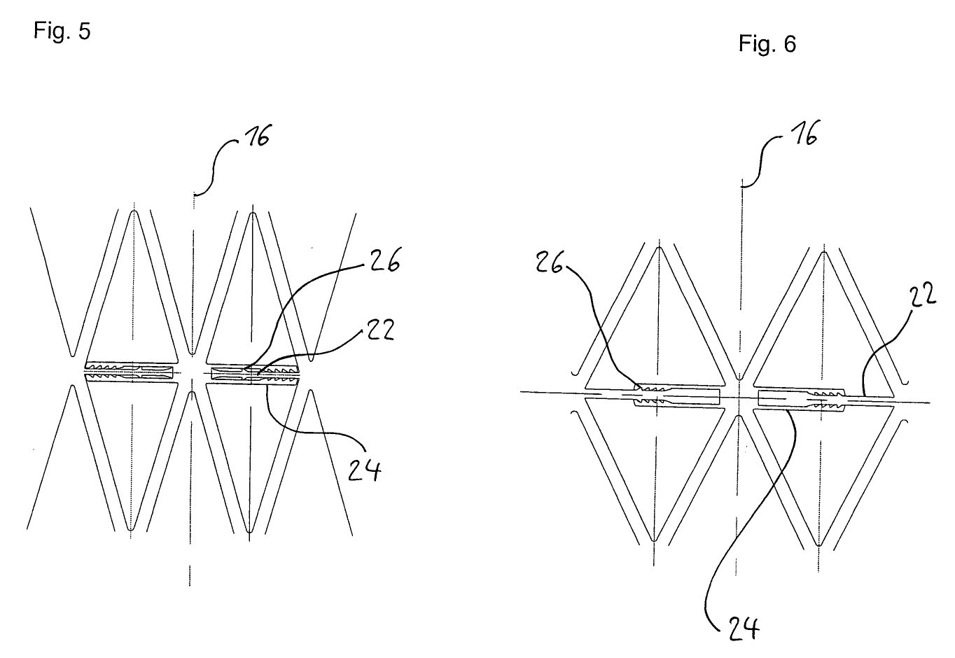

The many diamonds arranged alongside one another in the circumferential direction of the stent 10, as they are illustrated in FIG. 4, lead to an enlargement of the circumference of the stent 10, upon expansion of said stent 10, and thus to the desired and defined cross section of flow in the lumen. Since in each case the coupling arrangement 36 in each individual diamond prevents further deformation of the diamond shape, the expanded overall form of the stent 10 is fixed and can no longer be changed to any appreciable extent. An undefined further expansion and/or compression of the stent 10 is thus reliably avoided. It is conceivable, however, to provide coupling arrangements 36 only in every second or more diamond shape (preferably substantially regularly in the circumferential direction of the stent). It is also conceivable to provide a large number of locking possibilities along the direction of movement of the nodes 14 toward one another, e.g., by providing a large number of catch lugs 26 and/or corresponding recesses 28 one after another, so that the stent 10 can be arranged expanded in different degrees or states of expansion. In other words, by providing coupling arrangements at different axial positions or degrees of expansion, it can be ensured that the stent can preferably be locked in different states of expansion (in which the nodes are moved toward one another by different distances X1 . . . Xn).

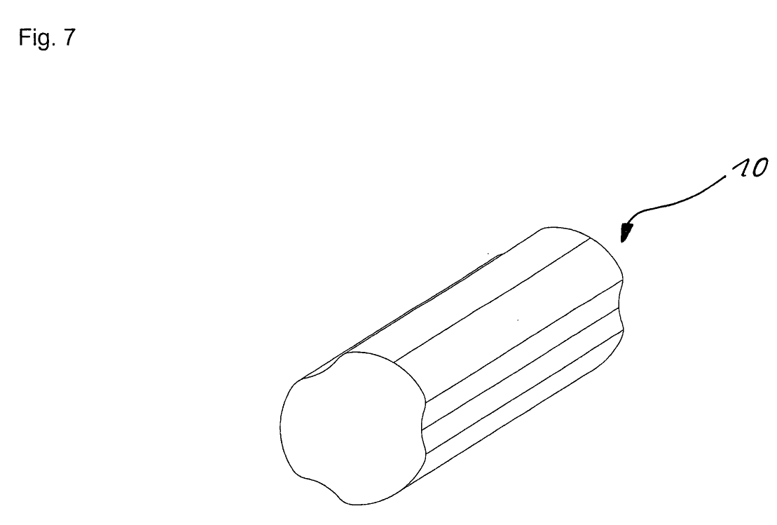

FIGS. 5 and 6 show a stent 10 where the plug elements 22, 24 are provided in the circumferential direction of the stent 10, i.e., substantially at right angles to the longitudinal axis 16 of the stent 10. In such a stent 10 the plug elements 22, 24 are moved away from each other during expansion of the stent 10, as illustrated in FIG. 6. In other words, with this stent 10, in the compressed state of the stent 10, the plug elements 22, 24 assume a converging position, as is illustrated in FIG. 5, and move away from each other when the stent 10 is expanded, as is illustrated in FIG. 6.

Accordingly, the catch lug(s) 26 of the male plug element 22 and the recess(es) 28 of the female plug element 24 are formed such that they become locked when they are moved away from each other. When a multiplicity of catch lugs 26 and/or recesses 28 exists, a multi-stage snap-fit connection can be formed.

The plug elements 22, 24 can also be used together, both in the circumferential direction and in the axial direction. Furthermore, shapes of an expanded stent 10 that deviate from a circular shape can be formed by varying the design the plug elements, as is illustrated in FIG. 7.

The stent 10 shown in FIG. 7 comprises three constrictions on its circumference in the expanded state, which constrictions may be formed by different plug elements 22, 24. It is conceivable, however, to form also other shapes of the expanded stent 10 through different plug elements 22, 24, for example an elliptic shape, a cone shape, a wave shape etc.

The stent 10 according an embodiment thereof can be produced both from tubular material and also from flat material. In the latter case the stent subsequently being rolled up, welded and/or finished. The stent 10 can also be produced by means of laser cutting, laser removal, photochemical etching and/or erosion. The stent 10 can also be produced with the stent structure in an at least partially expanded form, and the stent is then reduced in size to a compressed shape for insertion into the catheter, e.g., before it is later expanded at least partially in the body again.

Alternatively, the present invention can be used particularly effectively in balloon-expanded stents made of stainless steel, tantalum, niobium, cobalt alloys and other materials, for example polymers, self-degradable materials (e.g., lactic acid materials or derivatives), and in stents made of nitinol (nickel-titanium alloys) and/or of other self-expandable materials or shape-memory materials.

Among other uses, the stent or spreader or support structure disclosed above can be used for stabilizing vessels, in particular blood vessels, or as a tracheal stent, bronchial stent, transhepatic portosystemic shunt, transhepatic intravenous portosystemic shunt (TIPS), bile duct stent and/or embolic protective device. The stent or spreader or support structure can also be used as a support stent for implants, in particular for a heart valve, a venous valve, a vena cava filter, a prostatic sphincter body and/or as anti-reflux stent (stomach valve).

The invention has been described above and, obviously, modifications and alternations will occur to others upon a reading and understanding of this specification. In addition, the method of use described above is not limited to the order in which the steps above are recited. The claims as follows are intended to include all modifications and alterations insofar as they come within the scope of the claims or the equivalent thereof.

Having thus described the invention, I claim:

1. A stent for inserting and expanding in a lumen, the stent comprising:
at least two portions of a stent structure that are moveable substantially away from each other upon expansion of the stent; and
at least one coupling arrangement disposed between the two portions wherein after at least one predefined movement of the two portions, the two portions are coupled to each other in such a way that movement of the two portions substantially towards each other is avoided.

2. The stent of claim 1, wherein further movement of the two portions is substantially avoided.

3. The stent of claim 2, wherein the two portions comprises a first portion and a second portion and said at least one coupling arrangement comprises a first and a second coupling member.

4. The stent of claim 3, wherein movement of the first and second portions toward and away from one another is substantially avoided.

5. The stent of claim 4, wherein the first coupling member is configured with a male plug and the second coupling member is configured with a female plug.

6. The stent of claim 5, wherein the first and second coupling members are configured with at least one guide element with which the male plug element is guided into the female plug element when the first and second portions are moved toward one another.

7. The stent of claim 3, wherein the first and second coupling members are formed with two bridge halves that are oriented substantially coaxially and have mutually facing end areas.

8. The stent of claim 3, wherein first and second coupling members are formed with two bridge halves that are oriented substantially coaxially and which have a guide rod and a guide groove arranged as guide elements at mutually facing end areas.

9. The stent of claim 3, wherein the first and second coupling members are guided relative to one another by a guide element in a non-expanded state of the stent.

10. The stent of claim 3, wherein the first and second coupling members are configured with at least one hook element that catches when the first and second portions are moved toward one another.

11. The stent of claim 3, further including at least four support bridges pivotably connected with one another at nodes and, in an expanded state of the stent, form a closed ring shape, wherein the first and second coupling members are disposed.

12. The stent of claim 2, wherein the coupling arrangement extends substantially in the direction of a longitudinal axis of the stent.

13. The stent of claim 2, wherein the coupling arrangement extends substantially in the circumferential direction of the stent.

14. The stent of claim 1, wherein the stent comprises at least one coupling arrangement extending substantially in the circumferential direction of the stent and at least one coupling arrangement extending substantially in the direction of a longitudinal axis of the stent.

15. The stent of claim 1, used as support for a heart valve.

16. The stent of claim 1 used as support for at least one of a venous valve, a vena cava filter, a prostatic sphincter body and an anti-reflux stent.

17. A method for applying a stent for at least one of insertion and expansion in a lumen, the stent comprising at least two portions of a stent body that are moveable substantially towards each other when expanding the stent, the method comprising:
expanding the stent a predefined movement of the two portions; and
coupling the two portions to each other by means of a coupling arrangement in such a way that movement of the two portions away from each other is substantially avoided and further movement of the two portions towards each other is substantially avoided.
